(19) United States
(12) Patent Application Publication
(10) Pub. No.: US 2003/0108894 A1
(43) Pub. Date: Jun. 12, 2003

(54) Novel polypeptide-atp-dependent helicase protein 68 and the polynucleotide encoding said polypeptide

(76) Inventors: Yumin Mao, Shanghai (CN); Yi Xie, Shanghai (CN)

Correspondence Address: Jonathan S Caplan, Kramer Levin Naftalis & Frankel, 919 Third Avenue, New York, NY, 10022, US

(21) Appl. No.: 10/168,836

(22) PCT Filed: Dec. 18, 2000
(86) PCT No.: PCT/CN00/00581

(51) Int. Cl. C12Q 1/68; C07H 21/04; C12P 21/02; C12N 5/06; C12N 9/22; A61K 48/00

(52) U.S. Cl. 435/6; 435/69.1; 435/320.1; 435/325; 435/199; 514/44; 536/23.2

(57) ABSTRACT

The invention discloses a new kind of ATP-dependent helicase protein 68 and the polynucleotide encoding said polypeptide and a process for producing the polypeptide by recombinant methods. It also discloses the method of applying the polypeptide for the treatment of various kinds of diseases, such as cancer, hemopathy, HIV infection, immune diseases and inflammation. The antagonist of the polypeptide and therapeutic use of the same is also disclosed. In addition, it refers to the use of polynucleotide encoding said ATP-dependent helicase protein 68.

### FIELD OF INVENTION

The invention relates to the field of biotechnology. In particular, the invention relates to a novel polypeptide, designated ATP-dependent helicase protein 68, and a polynucleotide sequence encoding said polypeptide. The invention also relates to methods for the preparation and application of said polynucleotide and polypeptide.

### TECHNICAL BACKGROUND OF THE INVENTION

In organisms' bodies, there is a kind of proteins that have similar structural characteristics in eukaryotes and in prokaryotes, their functions inside organisms are ATP-dependent. These proteins distribute extensively in organisms' bodies, found in all sorts of organisms, they constitute a big protein family—ATP-dependent helicase superfamily.

In many activities of cells, the regulations of RNA structure such as splicing of precursor RNA, assembling of spliceosome, protein translation, are all required regulatory procedures. These processes are often regulated by various different helicases. The helicases can be detected in many biological systems in which RNA plays key roles. They exist widely in various tissues and organs of organisms from prokaryotes (virus included) to lower and higher organisms, they participate in nucleus and mitochondrion fission, RNA editing, rRNA modification, transcription initiation, transportation of nuclear mRNA and mRNA degradation. The helicases are considered as an important factor in cell development and differentiation, some of them play roles in the processes of virus ssRNA translation and replication [Arri Eisen, John C. Lucchesi, Bioessays, 1998, 20: 634-641]. They supply an effective approach to diagnosis, prevention and treatment for cancer, nervous system diseases and immune system diseases.

Studies show that members of ATP-dependent helicase superfamily contain many conservative sequences motifs, some of the motifs are specific to certain family members, whereas others exist not only in members of this family but also in some ATP-binding proteins. One of the sequence motif is “D-E-A-D” box, existing in B motif of ATP-binding proteins. Other members of the helicase superfamily contain a “D-E-A-H” box, which is specific to the helicase family, this box has a His residue which replaces Asn in “D-E-A-D” box.

“D-E-A-D” box and “D-E-A-H” box both exist in B structural motif of ATP-dependent helicase, B structural motif of all of these family members contain the following identical sequence fragment: [LIVMF] (2)-D-E-A-D-[RKEN]-X-[LIVMFYGSTN];

This sequence fragment is a key regulatory site for ATP hydrolyses. Mutation of this motif will lead to inhibition of ATP hydrolyses, which will affect the enzymes' normal physiological functions. ATP-dependent helicase is an important regulatory factor in cell development and differentiation, often involved in occurrences of nervous system diseases, immune system diseases, tumors, cancers and so on.

Because that the ATP-dependent helicase protein 68 mentioned above has important functions in important biological processes, and it is believed that these regulatory processes involve large quantity of proteins, identifications of more molecules of ATP-dependent helicase protein 68 involved in those process are needed, especially identifications of their amino acid sequences. The isolation of the new gene encoding ATP-dependent helicase protein 68 provides foundation for studying the functions of this protein in healthy and ill subjects. The protein in this invention may become a foundation for diagnoses and treatment for various diseases, so it is important to identify the DNA coding sequence for this protein.

### DISCLOSURE OF THE INVENTION

An object of the present invention is to provide an isolated novel polypeptide, designated ATP-dependent helicase protein 68, and the fragments, analogs and derivatives thereof.

Another object of the present invention is to provide a polynucleotide which encodes said polypeptide.

Another object of the present invention is to provide a recombinant vector containing a polynucleotide encoding ATP-dependent helicase protein 68.

Another object of the present invention is to provide a genetically engineered host cells containing a polynucleotide encoding ATP-dependent helicase protein 68.

Another object of the present invention is to provide a method for producing ATP-dependent helicase protein 68.

Another object of the present invention is to provide antibodies against ATP-dependent helicase protein 68.

Another object of the present invention is to provide mimetics, antagonists, agonists, and inhibitors of ATP-dependent helicase protein 68.

Another object of the present invention is to provide methods for the diagnosis and treatment of the diseases in which abnormalities in ATP-dependent helicase protein 68 expression are associated.

### BRIEF DESCRIPTIONS OF THE INVENTION

The present invention involves an isolated polypeptides of human origin, which comprises the amino acid sequence of SEQ ID NO: 2, or its conservative mutants, active fragments, active derivatives or analogues. Preferably, the polypeptide has the amino acid sequence of SEQ ID NO: 2.

The present invention also involves an isolated polynucleotide, which comprises a nucleotide sequence or its variant selected from the group consisting of (a) the polynucleotide encoding a polypeptide comprising the amino acid sequence of SEQ ID NO: 2; (b) a polynucleotide complementary to the polynucleotide (a); and (c) a polynucleotide that shares at least 70% homology to the polynucleotide (a) or (b). Preferably, said nucleotide sequence is selected from the group consisting of (a) the sequence of position 176-2035 in SEQ ID NO: 1; and (b) the sequence of position 1-2248 in SEQ ID NO: 1.

The present invention also involves: a kind of vector containing the polynucleotides of said invention, especially an expression vector; a kind of host cell genetically engineered with the said vector and the host cell can be used for transformation, transduction and transfection; a method for the production of the said invention polypeptide through the process of host cell cultivation and expession product collection.

The said invention also involves a kind of antibody which could specifically bind to the said invented polypeptide.

The said invention also includes a method for the screening of compounds which could simulate, activate, antagonize, or inhibit the activity of ATP-dependent helicase protein 68, and the compounds obtained by the said method.

The said invention also includes a method for in vitro assay of the diseases or disease susceptibility related to the abnormal expression of ATP-dependent helicase protein 68. The method involves mutation detection of the said polypeptide or its encoding polynucleotide sequence, or the quantitative determination or biological activity assay of the said invented polypeptide in biological samples.

The said invention also includes a kind of compound drug which includes the said invented polypeptide, its mimic, its agonists, its antagonist, its inhibitor, and acceptable carriers in medicine.

The said invention also includes application of said invented polypeptide and/or its polynucleotide for drug development to treat cancers, developmental diseases, immune diseases, or other diseases caused by abnormal expression of ATP-dependent helicase protein 68.

Other aspects of the present invention are apparent to those skilled in the art in view of the disclosure.

### BRIEF DESCRIPTION OF THE DRAWINGS

The following drawings are provided to illustrate embodiments of the invention, not to limit the scope of the invention as defined by the claims.

FIG. 1 is a homology comparison between helicase protein68 (aa331-379, 49 aa containing structural domain) of the present invention and amino acid sequence of ATP-dependent helicase family protein. The top sequence is ATP-dependent helicase protein 68, the one beneath is the structural domain of ATP-dependent helicase family protein. “|”, “:” and “,” indicate that the probability of the occurrence of the same amino acid in both sequences at the same position gradually decreases.

FIG. 2 shows the image of SDS-PAGE of the isolated ATP-dependent helicase protein 68, which has a molecular weight of 68 kDa. The isolated protein band is marked with an arrow.

### DISCLOSURE OF THE INVENTION

The terms used in this specification and claims have the following meanings, except the special descriptions.

“Nucleotide sequence” refers to oligonucleotide, nucleotide, or polynucleotide and polynucleotide fragments. It also refers to gene groups or synthetic DNA or RNA, which could be single-stranded or double-stranded, and could represent sense strand or antisense strand. Similarly, the term “amino acid sequence” refers to oligopeptide, peptide, polypeptide, or protein sequence and protein fragments or parts. When the “amino acid sequence” in said invention is related to the sequence of a natural protein, the amino acid sequence of said kind of “peptide” or “protein” will not be limited to be identical to the complete sequence of that natural protein.

“Mutant” of a protein or polynucleotide refers to the amino acid sequence with one or several amino acid changed, or its encoding polynucleotide sequence with one or several nucleotides changed. Such changes include deletion, insertion, or substitution of amino acids in the animo acid sequence, or of nucleotides in the polynucleotide sequence. These changes could be conservative and the substituted amino acid has similar structure or chemical characteristics as the original one, just as the substitution of Ile with Leu. Changes also could be not conservative, just as the substitution of Ala with Trp.

“Deletion” refers to the deletion of one or several amino acids in the amino acid sequence, or of one or several nucleotides in the nucleotide sequence.

“Insertion” or “addition” refers to the addition of one or several amino acids in the amino acid sequence, or of one or several nucleotides in the nucleotide sequence, compared to the naturally occurring molecule. “Substitution” refers to the change of one or several amino acids, or of one or several nucleotides, into different ones in the same position.

“Biological activity” refers to a protein with structure, regulation or biochemical functions of a natural molecule. Similarly, the term “immunologic competence” refers to the ability of natural, recombinant, or synthetic proteins to induce a specific immunologic response, or to bind to a specific antibody in the appropriate kinds of animals or cells.

“Agonist” refers to the kind of molecule which could regulate the activity of ATP-dependent helicase protein 68 by binding and changing it. Agonists can be proteins, nucleotides, carbohydrates or any other molecules which could bind to ATP-dependent helicase protein 68.

“Antagonist” or “inhibitor” refers to the kind of molecule which could repress or downregulate the biological activity or immune activity of ATP-dependent helicase protein 68 when binding to it. Antagonists or inhibitors can be proteins, nucleotides, carbohydrates or any other molecules which could bind to ATP-dependent helicase protein 68.

“Regulation” refers to the functional change of ATP-dependent helicase protein 68, including up-regulation or reduction of the protein activity, changes in binding specificity, changes of any other biological characteristics, functional or immunologic characteristics of ATP-dependent helicase protein 68.

“Mainly pure” refers to the condition of mostly without any other naturally related proteins, lipids, saccharides, or other substances. Technicians in this field can purify ATP-dependent helicase protein 68 by standard protein purification techniques. Mainly pure ATP-dependent helicase protein 68 proteins produce a single main band in denaturing polyacrylamide gel. The purity of ATP-dependent helicase protein 68 can be analyzed by amino acid sequence analysis.

“Complementary” or “complementation” refers to the natural conjugation of polynucleotides by base pairing under the condition of allowed salt concentration and temperature. For instance, the sequence “C-T-G-A” could bind its complementary sequence “G-A-C-T”. The complementation between two single-stranded molecules could be partial or complete. Complementary degrees between nucleotide strands has obvious influence on hybridization efficiency and intensity of the nucleotide strands.

“Homology” refers to the complementary degree which can be partially or completely homologous. “Partial homology” refers to a kind of partially complementary sequence to a target nucleotide and the sequence could at least partially repress the hybridization between a completely complementary sequence and the target nucleotide. Repression of the hybridization could be assayed by hybridization (Southern blot or Northern blot) under a lower stringency condition. Mainly complementary sequence or hybrid probe could compete with the completely complementary sequence and repress its hybridization with the target sequence under a lower stringency condition. This effect does not mean that nonspecific binding is allowed under a lower stringency condition, because specific or selective reaction is still required for hybridization under a lower stringency condition.

“Identity percentage” refers to the percentage of sequence identity or similarity when two or several kinds of amino acid or nucleotide sequences are compared. Identity percentage could be determined by computation method such as MEGALIGN program (Lasergene software package, DNASTAR, Inc., Madison Wis.). MEGALIGN software program can compare two or several sequences with different kinds of methods such as Cluster method (Higgins, D. G. and P. M. Sharp (1988) Gene 73:237-244). Cluster method examines the distance between all sequence pairs and arranges the sequences into clusters. Then the clusters are partitioned by pairs or groups. The identity percentage between two amino acid sequences such as sequence A and B can be calculated by the following equation:

$$\frac{Number of paired residues between sequence A and B}{\begin{matrix}Residue number of sequence A - number of spacing residues \\ in sequence A - number of spacing residues in sequence B\end{matrix}} \times 100$$

Identity percentage between nucleotide sequences can also be determined by Cluster method or other well-known methods in this field, such as Jotun Hein method (Hein J., (1990) Methods in emzumology 183:625-645).

“Similarity” refers to the degrees of identical or conservative substitution of amino acid residues in corresponding sites of the amino acid sequences compared to each other. Amino acids for conservative substitution are: negative charged amino acids including Asp and Glu; positive charged amino acids including Lys and Arg; amino acids with similar wate-affinity and with non-charged head chains including the following groups: Leu, Ile and Val; Gly and Ala; Asn and Gln; Ser and Thr; Phe and Tyr.

“Antisense” refers to the nucleotide sequences complementary to a specific DNA or RNA sequence. “Antisense strand” refers to the nucleotide strand complementary to the “sense strand”.

“Derivative” refers to HFP or the chemically modified nucleotides encoding it. The kind of modified chemicals can be derived from replacement of the hydrogen atom with Alkyl, Acyl, or Amino groups. The nucleotide derivative can encode peptide retaining the major biological characteristics of the natural molecule.

“Antibody” refers to the intact antibody or its fragments such as Fa, F(ab′)₂ and Fv, and it can specifically bind to the epitopes of ATP-dependent helicase protein 68.

“Humanized antibody” refers to an antibody which has its amino acid sequence in non-antigen binding region replaced to mimic human antibody and still retains the original binding activity.

The term “isolated” refers to the removal of a substance out of its original environment (for instance, if it's naturally produced, original environment refers to its natural environment). For example, a naturally produced polynucleotide or a polypeptide in a living organism means it has not been “isolated”. While the separation of the polynucleotide or a polypeptide from its coexisting materials in natural system means it is “isolated”. Such a polynucleotide may be part of a vector. This polynucleotide or polypeptide may also be part of a compound. Since the vector or compound is not part of its natural environment, the polynucleotide or polypeptide is still “isolated”.

As used herein, the term “isolated” refers to a substance which has been isolated from the original environment. For naturally occurring substances, the original environment is the natural environment. For example, polynucleotides and polypeptides in a naturally occurring state in viable cells are not isolated or purified. However, if the same polynucleotides and polypeptides have been isolated from other components naturally accompanying them, they are isolated or purified.

As used herein, “isolated ATP-dependent helicase protein 68” means that ATP-dependent helicase protein 68 does not essentially contain other proteins, lipids, carbohydrates or any other substances associated therewith in nature. Those skilled in the art can purify ATP-dependent helicase protein 68 by standard protein purification techniques. The purified polypeptide forms a single main band on a non-reductive PAGE gel. The purity of ATP-dependent helicase protein 68 can be determined by amino acid sequence analysis.

The present invention provides a novel polypeptide designated ATP-dependent helicase protein 68, which mainly comprises the amino acid sequence shown in SEQ ID NO: 2. The polypeptide of the invention may be a recombinant polypeptide, a natural polypeptide, or a synthetic polypeptide, preferably a recombinant polypeptide. The polypeptide of the invention may be a purified natural product or a chemical synthetic product. Alternatively, it may be produced from prokaryotic or eukaryotic hosts, such as bacterial, yeast, higher plant, insect, and mammalian cells, using recombinant techniques. Depending on the host used in the protocol of recombinant production, the polypeptide of the invention may be glycosylated or non-glycosylated. The polypeptide of the invention may or may not contain the starting Met residue.

The invention further comprises fragments, derivatives and analogs of ATP-dependent helicase protein 68. As used in the invention, the terms “fragment”, “derivative” and “analog” mean that the polypeptide essentially retains the same biological function or activity of ATP-dependent helicase protein 68. The fragments, derivatives or analogs of the polypeptide may be (i) one in which one or more of the amino acid residues are substituted with conserved or non-conserved amino acid residues (preferably conserved amino acid residues) and such substituted amino acid residues may or may not be one encoded by the genetic code; or (ii) one in which one or more of the amino acid residues have their groups substituted by other groups, include substituent groups; or (iii) one in which the mature polypeptide is fused with another compound, such as a compound to increase the half-life of the polypeptide (for example, polyethylene glycol); or (iv) one in which additional amino acids are fused to the mature polypeptide, such as a leader or secretory sequence or a sequence which is employed for purification of the mature polypeptide or a precursor sequence. Such fragments, derivatives and analogs are deemed to be within the scope of a person skilled in the art from the teachings herein.

The invention also provides an isolated nucleic acid or polynucleotide which comprises a polynucleotide encoding an amino acid sequence of SEQ ID NO: 2. The polynucleotide sequence of the invention includes the nucleotide sequence of SEQ ID NO: 1. The polynucleotide of the invention was identified in a human embryonic brain cDNA library. Preferably, it comprises a full-length polynucleotide sequence of 2248 bp, whose ORF (176-2035) encodes 619 amino acids. The polypeptide has character sequence of ATP-dependent helicase family protein, it can be concluded that this novel ATP-dependent helicase protein 68 has structures and functions ATP-dependent helicase family proteins representing.

The polynucleotide of the present invention may be in the form of DNA or RNA. If in the form of DNA it includes cDNA, genomic DNA, and synthetic DNA in single-stranded or double-stranded forms. DNA may be an coding strand or non-coding strand. The coding sequence for a mature polypeptide may be identical to the coding sequence shown in SEQ ID NO: 1, or may be a degenerate sequence. As used herein, the term “degenerate sequence” means a sequence which encodes a protein or polypeptide comprising SEQ ID NO: 2 and which has a nucleotide sequence different from the sequence of the coding region in SEQ ID NO: 1.

The polynucleotide encoding the mature polypeptide of SEQ ID NO: 2 includes those encoding only the mature polypeptide, those encoding the mature polypeptide plus various additional coding sequences, the coding sequence for the mature polypeptide (and optional additional coding sequence) plus the non-coding sequence.

The term “polynucleotide encoding the polypeptide” includes polynucleotides encoding said polypeptide and polynucleotides comprising additional coding and/or non-coding sequences.

The invention further relates to variants of the above polynucleotides which encode a polypeptide having the same amino acid sequence, or a fragment, analog and/or derivative of said polypeptide. Variants of the polynucleotide may be naturally occurring allelic variants or non-naturally occurring variants. Such nucleotide variants include substitution, deletion, or insertion variants. As known in the art, an allelic variant may have a substitution, deletion, and insertion of one or more nucleotides without substantially changing the functions of the encoded polypeptide.

The present invention further relates to polynucleotides, which hybridize to the hereinabove-described sequences; that is, there is at least 50% and preferably at least 70% identity between the sequences. The present invention particularly relates to polynucleotides which hybridize to polynucleotides of the invention under stringent conditions. As herein used, the term “stringent conditions” means the following conditions: (1) hybridization and washing under low ionic strength and high temperature, such as 0.2×SSC, 0.1% SDS, 60° C.; or (2) hybridization after adding denaturants, such as 50% (v/v) formamide, 0.1% bovine serum/0.1% Ficoll, 42° C.; or (3) hybridization only when the homology of two sequences is at least 95%, preferably 97%. Further, the polynucleotide which hybridize to the hereinabove described polynucleotide encode a polypeptide which retains the same biological function and activity as the mature polypeptide of SEQ ID NO: 2.

The invention also relates to nucleic acid fragments which can hybridize to the hereinabove described sequences. As used in the present invention, the length of the “nucleic acid fragment” is at least more than 10 bp, preferably at least 20-30 bp, more preferably at least 50-60 bp, and most preferably at least 100 bp. The nucleic acid fragment can be used in amplification techniques of nucleic acid, such as PCR, so as to determine and/or isolate the polynucleotide encoding ATP-dependent helicase protein 68.

The polypeptide and polynucleotide of the present invention are preferably in the isolated forms, and preferably purified to be homogenous.

In the present invention, the specific nucleic acid sequence encoding ATP-dependent helicase protein 68 can be obtained in various ways. For example, the polynucleotide is isolated by hybridization techniques well-known in the art, which include, but are not limited to 1) the hybridization between a probe and genomic or cDNA library so as to select a homologous polynucleotide sequence, and 2) antibody screening of an expression library so as to obtain polynucleotide fragments encoding polypeptides having common structural features.

In the present invention, DNA fragment sequences may be further obtained by the following methods: 1) isolating double-stranded DNA sequence from genomic DNA; and 2) chemical synthesis of DNA sequences so as to obtain the double-stranded DNA.

Among the above mentioned methods, the isolation of genomic DNA is least frequently used. A commonly used method is the direct chemical synthesis of a DNA sequence. A more frequently used method is the isolation of a cDNA sequence. A standard method for isolating the cDNA of interest is isolating mRNA from donor cells that highly express said gene, followed by reverse transcription of mRNA to form a plasmid or phage cDNA library. There are many established techniques for extracting mRNA and kits are commercially available to do so (Qiagene). Conventional methods can be used to construct cDNA library (Sambrook, et al., Molecular Cloning, A Laboratory Manual, Cold Spring Harbor Laboratory. New York, 1989). cDNA libraries are also commercially available. For example, Clontech Ltd. has various cDNA libraries. When PCR is used, even extremely small amounts of expression products can be cloned.

Conventional methods can be used to screen for polynucleotides of the present invention from a cDNA library. These methods include, but are not limited to, (1) DNA-DNA or DNA-RNA hybridization; (2) the emergence or loss of the function of the marker-gene; (3) the determination of the level of ATP-dependent helicase protein 68 transcripts; (4) the determination of protein product of gene expression by immunology methods or the biological activity assays. The above methods can be used alone or in combination.

In method (1), the probe used in hybridization may be homologous to any portion of the polynucleotides of the invention. The length of probe is typically at least 10 nucleocides, preferably at least 30 nucleocides, more preferably at least 50 nucleocides, and most preferably at least 100 nucleotides. Further, the length of the probe is usually less than 2000 nucleotides, and preferably less than 1000 nucleotides. The probe employed here is usually the DNA sequence chemically synthesized on the basis of the sequence information of the present invention. Of course, the gene of the invention itself or its fragment can be used as a probe. The labels for DNA probe include, for example, radioactive isotopes, fluoresceins or enzymes such as alkaline phosphatase.

In method (4) referred to above, the detection of the protein products of ATP-dependent helicase protein 68 gene expression can be carried out by immunology methods, such as Western blotting, radioimmunoassay, and ELISA.

The method of amplification of DNA/RNA by PCR (Saiki, et al. Science 1985; 230:1350-1354) is preferably used to obtain the polynucleotide of the invention. Especially when it is difficult to obtain the full-length cDNA, the method of RACE (RACE-cDNA terminal rapid amplification) is preferably used. The primers used in PCR may be selected according to the polynucleotide sequence information of the invention disclosed herein, and can be synthesized by conventional methods. The amplified DNA/RNA fragments can be isolated and purified by conventional methods such as gel electrophoresis.

Sequencing of polynucleotide sequences of the gene of the invention or its various DNA fragments can be carried out by the conventional dideoxy sequencing method (Sanger et al. PNAS, 1977, 74: 5463-5467). Sequencing of polynucleotide sequences also can be carried out using commercially available sequencing kits. In order to obtain the full-length cDNA sequence, it is necessary to repeat the sequencing process. Sometimes, it is necessary to sequence the cDNA of several clones to obtain the full-length cDNA sequence.

The invention further relates to a vector comprising the polynucleotide of the invention, a genetically engineered host cell transformed with the vector of the invention or directly with the sequence encoding ATP-dependent helicase protein 68, and a method for producing the polypeptide of the invention by recombinant techniques.

In the present invention, the polynucleotide sequences encoding ATP-dependent helicase protein 68 may be inserted into a vector to form a recombinant vector containing polynucleotides of the invention. The term “vector” refers to a bacterial plasmid, bacteriophage, yeast plasmid, plant virus or mammalian virus such as adenovirus, retrovirus or any other vector known in the art. Vectors suitable for use in the present invention include, but are not limited to the T7 promoter-based expression vector for expression in bacteria (Rosenberg, et al., Gene, 56:125, 1987), the pMSXND expression vector for expression in mammalian cells (Lee and Nathans, J Bio. Chem., 263:3521, 1988) and baculovirus-derived vectors for expression in insect cells. Any plasmid or vector can be used to construct the recombinant expression vector as long as it can replicate and is stable in the host. One important feature of an expression vector is that the expression vector typically contains an origin of replication, a promoter, and a marker gene as well as translation regulatory components.

Methods known in the art can be used to construct an expression vector containing the DNA sequence of ATP-dependent helicase protein 68 and appropriate transcription/translation regulatory components. These methods include in vitro recombinant DNA techniques, DNA synthesis techniques and, in vivo recombinant techniques (Sambrook, et al. Molecular Cloning, a Laboratory Manual, Cold Spring Harbor Laboratory, New York, 1989). The DNA sequence is effectively linked to a proper promoter in an expression vector to direct the synthesis of mRNA. Exemplary promoters are lac or trp promoter of E. coli; PL promoter of λ phage; eukaryotic promoters including CMV immediate early promoter, HSV thymidine kinase promoter, early and late SV40 promoter, LTRs of retrovirus, and other known promoters which control gene expression in prokaryotic cells, eukaryotic cells or viruses. The expression vector may further comprise a ribosome-binding site for initiating translation, transcription terminator and the like. Transcription in higher eukaryotes is increased by inserting an enhancer sequence into the vector. Enhancers are cis-acting elements of DNA, usually from about 10 to 300 bp in length that act on a promoter to increase gene transcription level. Examples include the SV40 enhancer on the late side of the replication origin 100 to 270 bp, the polyoma enhancer on the late side of the replication origin, and adenovirus enhancers.

Further, the expression vector preferably comprises one or more selective marker genes to provide a phenotype for the selection of the transformed host cells, e.g., the dehydrofolate reductase, neomycin resistance gene and GFP (green fluorescent protein) for eukaryotic cells, as well as tetracycline or ampicillin resistance gene for E. coli.

Those skilled in the art may select appropriate vectors and transcriptional regulatory elements, e.g., promoters, enhancers, and selective marker genes.

In the present invention, a polynucleotide encoding ATP-dependent helicase protein 68 or recombinant vector containing said polynucleotide can be transformed or transfected into host cells to construct genetically engineered host cells containing said polynucleotide or said recombinant vector. The term “host cell” means prokaryote, such as bacteria; or lower eukaryote, such as yeast; or higher eukaryotic, such as mammalian cells. Representative examples include bacterial cells, such as E. coli, Streptomyces, Salmonella typhimurium; fungal cells, such as yeast; plant cells; insect cells such as Drosophila S2 or Sf9; animal cells such as CHO, COS or Bowes melanoma.

Transformation of a host cell with DNA sequences of the present invention or a recombinant vector containing said DNA sequences may be carried out by conventional techniques well known to those skilled in the art. When the host is prokaryotic, such as E. coli, competent cells, which are capable of DNA uptake, can be prepared from cells harvested after the exponential growth phase and subsequently treated by the CaCl₂ method using procedures well known in the art. Alternatively, MgCl₂ can be used. Transformation can also be carried out by electroporation, if desired. When the host is an eukaryote, the following transfection methods can be employed: calcium phosphate precipitation, conventional mechanical procedures such as micro-injection, electroporation, or liposome-mediated packaging.

The recombinant ATP-dependent helicase protein 68 can be expressed or produced by conventional recombinant DNA technology (Science, 1984; 224:1431), using polynucleotide sequences of the invention. The steps generally include:

(1) transfecting or transforming the appropriate host cells with polynucleotides (or variants) encoding ATP-dependent helicase protein 68 of the invention or the recombinant expression vector containing said polynucleotides;

(2) culturing the host cells in an appropriate medium; and

(3) isolating or purifying the protein from the medium or cells.

In Step (2) above, depending on the host cells used, the medium for cultivation can be selected from various conventional mediums. The host cells are cultured under a condition suitable for growth until the host cells grow to an appropriate cell density. Then, the selected promoter is induced by appropriate means (e.g., temperature shift or chemical induction) and cells are cultured for an additional period.

In Step (3), the recombinant polypeptide may be included in the cells, or expressed on the cell membrane, or secreted out of the cell. If desired, physical, chemical and other properties can be utilized in various isolation methods to isolate and purify the recombinant protein. These methods are well-known to those skilled in the art and include, but are not limited to conventional renaturation treatment, treatment by a protein precipitant (such as salt precipitation), centrifugation, cell lysis by osmosis, sonication, supercentrifugation, molecular sieve chromatography or gel chromatography, adsorption chromatography, ion exchange chromatagraphy, HPLC, and any other liquid chromatography, or any combination thereof.

The polypeptide of the invention and antagonists, agonists and inhibitors thereof can be directly used for the treatment of diseases, e.g., various malignant tumors or cancers, dermatitis, inflammation, adrenoprival disease and HIV infection and immune system diseases.

ATP-dependent helicase superfamily is a very big protein family. In many activities of cells, such as fission of nucleus and mitochondrion, editing of RNA, processing of rRNA, transcription initiation, transportation of nuclear mRNA and degradation of mRNA, the ATP-dependent helicase plays a key role. ATP-dependent helicase family contains the characteristic structural sequence “D-E-A-D” box and “D-E-A-H” box. The helicase is considered as an important factor in cell development and diversity, some on processes of virus ssRNA translation and replication. [Arri E, et al., 1998].

It is therefore concluded that abnormal expression of specific “D-E-A-D” box and “D-E-A-H” box (characteristic structural sequence of ATP-dependent helicase family) will lead to abnormal function of the polypeptide by the present invention which contains “D-E-A-D” box and “D-E-A-H” box (characteristic structural sequence of ATP-dependent helicase family). Then it will lead to cell rRNA, mRNA and other RNA process and edit abnormally, and also lead to abnormal fission of nucleus and mitochondrion, which will cause cells differentiate and replicate abnormally and many related diseases occur, such as tumors, embryogenesis disorders, growth handicap diseases and so on.

As discussed above, abnormal expression of ATP-dependent helicase 68 by the present invention will lead to all kinds of diseases, especially various tumors, embryogenesis disorders, growth handicap diseases, these diseases include but not limited:

Canceration of various tissues: gastric carcinoma, hepatoma, lung cancer, gullet cancer, galactophore cancer, leucocythemia, lymphoma, tumor of thyroid, hysteromyoma, ovarian tumor, neuroblastoma, astrocytoma, ependymocytoma, neuroglioma, carcinoma of colon, malignant histiocytosis, melanoma, teratoma, sarcoma, adrenal cancer, carcinoma of urinary bladder, osteocarcinoma, osteosarcoma, myeloma, encephaloma, metrocarcinoma, endometrial carcinoma, carcinoma of gallbladder, thymic tumor, tumor of sinus of nose, carcinoma, laryngocarcinoma, tumor of trachea, mesothelioma of pleura, fibroma, adipoma, liposarcoma, leiomyoma.

Growth handicap diseases: mental retardation, cerebral paralysis, brain eccyliosis, family cranial nerves hypoplasia syndrome, strabismus, skin and fat and muscle hypogenetic syndromes (congenita lcutis relaxation), prematureageing, congenital cornifing badness, various metabolic deficient diseases (various amino-acid metabolic deficient diseases), cretinism, pygmyism, sexual hypoevolution.

Embryogenesis disorders: congenital abortion, palatoschisis, limbs and trunk differentiation obstruction, congenital exomphalocele, hyaline membrane disease, congenital lung sac turgescence, atelectasis, polycysticdisease, double ureter, umbilical urinary fistula, cryptorchidism, congenital inguinalhernia, double uterus, ankylocolpos, androgyny, atrial septal defect, interventricular septal defect, abnormal arterial sepration, patent ductus arteriosus, aortarctia, pulmonary stenosis, neural canal defect, congenital waterhead coloboma iridis, congenital cataract, congenital glaucoma, congenital deafness.

Abnormal expression of ATP-dependent helicase68 by the present invention will generate some hereditary, haematic diseases, immune system diseases and so on.

The polypeptide by the present invention and its antagonist, agonist and inhibitor can be directly used for treatments of diseases, for example, it can treats various diseases, especially various tumors, embryogenesis disorders, growth handicap diseases, some hereditary, haematic diseases, immune system diseases and so on.

The invention also provides methods for screening compounds so as to identify an agent which enhances ATP-dependent helicase protein 68 activity (agonists) or inhibits ATP-dependent helicase protein 68 activity (antagonists). The agonists enhance the biological functions of ATP-dependent helicase protein 68 such as stimulating cell proliferation, while the antagonists prevent and cure the disorders associated with excess cell proliferation, such as various cancers. For example, in the presence of an agent, mammalian cells or membrane preparations expressing ATP-dependent helicase protein 68 can be incubated with labeled ATP-dependent helicase protein 68 to determine the ability of the agent to enhance or repress the interaction.

Antagonists of ATP-dependent helicase protein 68 include antibodies, receptor deficient agents analogs and other compounds. The antagonists of ATP-dependent helicase protein 68 bind to ATP-dependent helicase protein 68 to eliminate or reduce its function, or inhibit the production of ATP-dependent helicase protein 68, or bind to the active sites of said polypeptide so that the polypeptide cannot function biologically.

When screening for antagonistic compounds, ATP-dependent helicase protein 68 may be added into a biological assay. It can be determined whether the compound is an antagonist by determining its effect on the interaction between ATP-dependent helicase protein 68 and its receptor. Using the same method as that for screening compounds, receptor deficient agents and analogs acting as antagonists can be selected. Polypeptide molecules capable of binding to ATP-dependent helicase protein 68 can be obtained by screening a random polypeptide library comprising various combinations of amino acids bound onto a solid matrix. Usually, ATP-dependent helicase protein 68 is labeled in the screening process.

The invention further provides a method for producing antibodies using the polypeptide, or its fragment, derivative, analog or cells comprising the polypeptide as an antigen. These antibodies may be polyclonal or monoclonal antibodies. The invention also provides antibodies against epitopes of ATP-dependent helicase protein 68. These antibodies include, but are not limited to, polyclonal antibodies, monoclonal antibodies, chimeric antibodies, single-chain antibodies, Fab fragments and fragments produced by a Fab expression library.

Polyclonal antibodies can be prepared by directly immunizing animals, such as rabbit, mouse, and rat, with ATP-dependent helicase protein 68. Various adjuvants, including but are not limited to Freund's adjuvant, can be used to enhance the immunization. The techniques for producing ATP-dependent helicase protein 68 monoclonal antibodies include, but are not limited to, the hybridoma technique (Kohler and Milstein. Nature, 1975, 256:495-497), the trioma technique, the human B-cell hybridoma technique, and the EBV-hybridoma technique. A chimeric antibody comprising a constant region of human origin and a variable region of non-human origin can be produced using methods well-known in the art (Morrison et al., PNAS, 1985, 81:6851). Furthermore, techniques for producing single-chain antibodies (U.S. Pat. No. 4,946,778) are also useful for preparing single-chain antibodies against ATP-dependent helicase protein 68.

Antibodies against ATP-dependent helicase protein 68 can be used in immunohistochemical methods to detect the presence of ATP-dependent helicase protein 68 in a biopsy specimen.

Monoclonal antibodies specific to ATP-dependent helicase protein 68 can be labeled by radioactive isotopes, and injected into a human body to trace the location and distribution of ATP-dependent helicase protein 68. The radioactively labeled antibodies can be used in a non-wounding diagnostic method for the determination of tumor location and metastasis.

Antibodies can also be designed as an immunotoxin targeting a particular site in the body. For example, a monoclonal antibody having high affinity to ATP-dependent helicase protein 68 can be covalently bound to bacterial or plant toxins, such as diphtheria toxin, ricin, and abrin. One common method is to challenge the amino group on the antibody with sulfydryl cross-linking agents, such as SPDP, and bind the toxin onto the antibody by interchanging the disulfide bonds. The hybrid antibodies can be used to kill ATP-dependent helicase protein 68-positive cells.

Antibodies of the present invention are useful for the therapy or prevention of disorders related to the ATP-dependent helicase protein 68. The appropriate amount of antibodies can be administered to stimulate or to block the production or activity of ATP-dependent helicase protein 68.

The present invention further provides diagnostic assays for quantitative and in situ measurement of ATP-dependent helicase protein 68 level. These assays are well known in the art and include FISH assays and radioimmunoassays. The level of ATP-dependent helicase protein 68 detected in an assay can be used to illustrate the importance of ATP-dependent helicase protein 68 in diseases and to determine the diseases associated with ATP-dependent helicase protein 68.

Polypeptides of the invention are useful in the analysis of a polypeptide profile. For example, polypeptides can be specifically digested by physical, chemical, or enzymatic means, and then analyzed by one, two or three dimensional gel electrophoresis, preferably by spectrometry.

The polynucleotide encoding ATP-dependent helicase protein 68 also have many therapeutic applications. Gene therapy technology can be used in the therapy of abnormal cell proliferation, development or metabolism, caused by the loss of ATP-dependent helicase protein 68 expression or the abnormal or non-active expression of ATP-dependent helicase protein 68. Recombinant gene therapy vectors, such as virus vectors, can be designed to express mutated ATP-dependent helicase protein 68 so as to inhibit the activity of endogenous ATP-dependent helicase protein 68. For example, one form of mutated ATP-dependent helicase protein 68 is a truncated ATP-dependent helicase protein 68 in which the signal transduction domain is deleted. Therefore, this mutated ATP-dependent helicase protein 68 can bind to the downstream substrate without the activity of signal transduction. Thus, recombinant gene therapy vectors can be used to cure diseases caused by abnormal expression or activity of ATP-dependent helicase protein 68. Expression vectors derived from viruses, such as for example retrovirus, adenovirus, adeno-associated virus, herpes simplex virus, parvovirus, can be used to introduce the ATP-dependent helicase protein 68 gene into the cells. Methods for constructing a recombinant virus vector harboring ATP-dependent helicase protein 68 gene are described in the literature (Sambrook, et al. supra). In addition, the recombinant ATP-dependent helicase protein 68 gene can be packed into liposome and then transferred into the cells.

The methods for introducing the polynucleotides into tissues or cells include directly injecting the polynucleotides into tissues in the body; or introducing the polynucleotides into cells in vitro with vectors, such as virus, phage, or plasmid, etc, and then transplanting the cells into the body.

Also included in the present invention are ribozymes and oligonucleotides, including antisense RNA and DNA, which inhibit the translation of the ATP-dependent helicase protein 68 mRNA. Ribozymes are enzyme-like RNA molecules capable of specifically cutting certain RNA. The mechanism is nucleic acid endo-cleavage following specific hybridization of the ribozyme molecules and the complementary target RNA. Antisense RNA and DNA as well as ribozymes can be prepared by using any conventional techniques for RNA and DNA synthesis, e.g., the widely used solid phase phosphite chemical method for oligonucleotide synthesis. Antisense RNA molecule can be obtained by the in vivo or in vitro transcription of the DNA sequence encoding said RNA, wherein said DNA sequence is integrated into the vector and downstream of the RNA polymerase promoter. In order to increase its stability, a nucleic acid molecule can be modified in many ways, e.g., increasing the length of the two flanking sequences, replacing the phosphodiester bond with the phosphothioester bond in the oligonucleotide.

The polynucleotide encoding ATP-dependent helicase protein 68 can be used in the diagnosis of ATP-dependent helicase protein 68 related diseases. The polynucleotide encoding ATP-dependent helicase protein 68 can be used to detect whether ATP-dependent helicase protein 68 is expressed, and whether the expression of ATP-dependent helicase protein 68 is abnormal in the case of diseases. For example, ATP-dependent helicase protein 68 DNA sequences can be used in hybridization with biopsy samples to determine the expression of ATP-dependent helicase protein 68. The hybridization methods include Southern blotting, Northern blotting and in situ blotting, etc., which are well-known and established techniques. Corresponding kits are commercially available. A part of or all of the polynucleotides of the invention can be used as probe and fixed on a microarray or DNA chip for analysis of differential expression of genes in tissues and for the diagnosis of genes. ATP-dependent helicase protein 68 specific primers can be used in RNA-polymerase chain reaction and for in vitro amplification to detect transcripts of ATP-dependent helicase protein 68.

Further, detection of mutations in ATP-dependent helicase protein 68 gene is useful for the diagnosis of ATP-dependent helicase protein 68-related diseases. Mutations of ATP-dependent helicase protein 68 include site mutation, translocation, deletion, rearrangement and any other abnormalities compared with the wild-type ATP-dependent helicase protein 68 DNA sequence. Conventional methods, such as Southern blotting, DNA sequencing, PCR and in situ blotting, can be used to detect a mutation. Moreover, mutations sometimes affect the expression of a protein. Therefore, Northern blotting and Western blotting can be used to determine indirectly whether the gene is mutated or not.

Sequences of the present invention are also valuable for chromosome identification. The sequence is specifically targeted to and can hybridize with a particular location on an individual human chromosome. There is a current need for identifying particular sites of genes on the chromosome. Few chromosome marking reagents based on actual sequence data (repeat polymorphism) are presently available for marking chromosomal location. The mapping of DNA to chromosomes according to the present invention is an important first step in correlating those sequences with genes associated with diseases.

Briefly, sequences can be mapped to chromosomes by preparing PCR primers (preferably 15-35 bp) from the cDNA. These primers are then used for PCR screening of somatic cell hybrids containing individual human chromosomes. Only those hybrids containing the human gene corresponding to the primer will yield an amplified fragment.

PCR mapping of somatic cell hybrids is a rapid procedure for assigning a particular DNA to a particular chromosome. Using the oligonucleotide primers of the present invention, sublocalization can be achieved with panels of fragments from specific chromosomes or pools of large genomic clones in an analogous manner. Other mapping strategies that can similarly be used to map chromosome locations include in situ hybridization, prescreening with labeled flow-sorted chromosomes and preselection by hybridization to construct chromosome specific-cDNA libraries.

Fluorescence in situ hybridization (FISH) of cDNA clones to a metaphase chromosomal spread can be used to provide a precise chromosomal location in one step. This technique is described in Verma et al., Human Chromosomes: a Manual of Basic Techniques, Pergamon Press, New York (1988).

Once a sequence has been mapped to a precise chromosomal location, the physical position of the sequence on the chromosome can be correlated with genetic mapping data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis.

Next, it is necessary to determine the differences in the cDNA or genomic sequence between affected and unaffected individuals. If a mutation is observed in some or all of the affected individuals but not in any normal individuals, then the mutation is likely to be the cause of the disease. Comparison of affected and unaffected individuals generally involves first looking for structural alterations in the chromosomes, such as deletions or translocations, that are visible from chromosome level, or detectable using PCR based on that cDNA sequence. With current resolution of physical mapping and genetic mapping techniques, a cDNA precisely localized to a chromosomal region associated with the disease could be one of 50 to 500 potential causative genes. (This assumes 1 megabase mapping resolution and one gene per 20 kb).

From the present invention, the polypeptides, polynucleotides and their mimetics, agonists, antagonists and inhibitors may be employed in combination with a suitable pharmaceutical carrier. Such a carrier includes but is not limited to water, glucose, ethanol, salt, buffer, glycerol, and combinations thereof. Such composites comprise a safe and effective amount of the polypeptide or antagonist, as well as a pharmaceutically acceptable carrier or excipient which has no influence on the effect of the drug. These composites can be used as drugs in disease treatment.

The invention also provides a pharmaceutical pack or kit comprising one or more containers filled with one or more of the ingredients of the pharmaceutical composites of the present invention. With such container(s) there may be a notice from a governmental agency that regulates the manufacture, use or sale of pharmaceuticals or biological products, the notice reflecting governmental approval for the manufacture, use or sale for human administration. In addition, the polypeptides of the invention may be employed in conjunction with other therapeutic compounds.

The pharmaceutical composites may be administered in a convenient manner, such as through topical, intravenous, intraperitoneal, intramuscular, subcutaneous, intranasal or intradermal routes. ATP-dependent helicase protein 68 is administered in an amount, which is effective for treating and/or prevention of the specific symptoms indicated. The amount and range of ATP-dependent helicase protein 68 administered to a patient will depend upon various factors, such as delivery methods, the subject's health, and the judgment of the skilled clinician.

Preferred Embodiments

The present invention is further illustrated by the following examples. It is appreciated that these examples are intended only to illustrate the invention, not to limit the scope of the invention. For experimental methods in the following examples, when detailed conditions are not elaborated, they are performed under routine conditions, e.g., those described by Sambrook, et al., in Molecule Cloning: A Laboratory Manual (New York: Cold Spring Harbor Laboratory Press, 1989), or as instructed by the manufacturers, unless otherwise specified.

### EXAMPLE 1

### Cloning of ATP-Dependent Helicase Protein 68 Gene

Total RNA from human embryonic brain was extracted by one-step method with guanidinium isocyanate/phenol/chloroform. The poly(A) mRNA was isolated from the total RNA with Quik mRNA Isolation Kit (Qiegene). cDNA was prepared by reverse transcription with 2 μg poly(A) mRNA. The cDNA fragments were inserted into the polyclonal site of pBSK(+) vector (Clontech) using Smart cDNA cloning kit (Clontech) and then transformed into DH5α bacteria to form the cDNA library. The 5′ and 3′ ends of all clones were sequenced with Dye terminate cycle reaction sequencing kit (Perkin-Elmer) and ABI 377 Automatic Sequencer (Perkin-Elmer). The cDNA sequences were compared with the public database of DNA sequences (Genebank) and the DNA sequence of one clone 0577G09 was found to be a novel DNA sequence. The inserted cDNA sequence of clone 0577G09 was dual-directionally sequenced with a series of synthesized primers. It was indicated that the full length cDNA contained in clone 0577G09 was 2248 bp (SEQ ID NO: 1) with a 1860 bp ORF located in positions 176 bp-2035 bp which encoded a novel protein (SEQ ID NO: 2). This clone was named pBS-0577G09 and the encoded protein was named ATP-dependent helicase protein 68.

### EXAMPLE 2

### Domain Analysis of cDNA Cloning

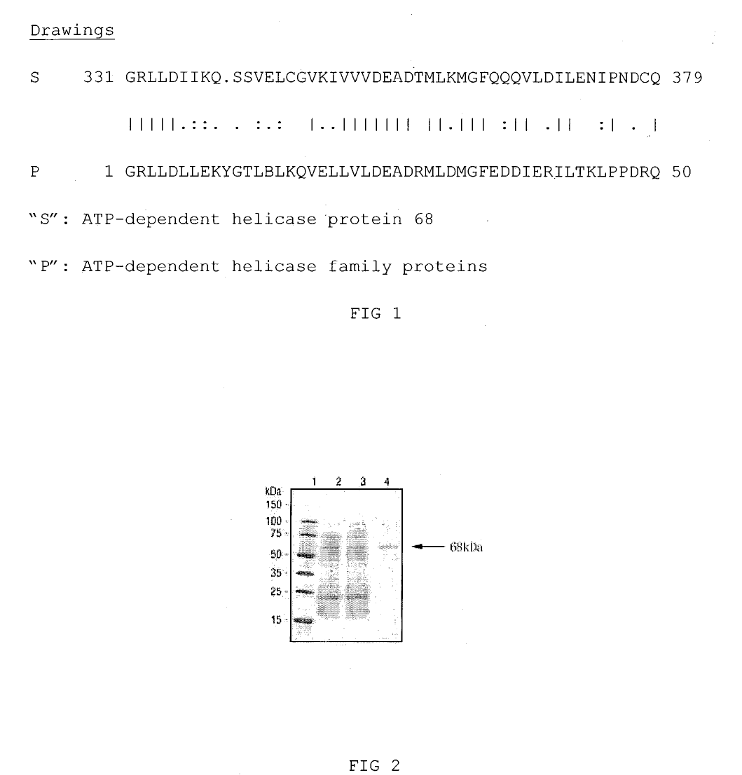

We analyze the sequences of ATP-dependent helicase protein 68 by the present invention and its encoding proteins with profile scan in GCG (Basic local alignment search tool) (Altschul, S F et al. J. Mol Biol. 1990; 215: 403-10) in Database Prosite, etc. ATP-dependent helicase68 by the present invention (aa37-83) has homology to ATP-dependent helicase family proteins, the result is showed in FIG. 1, the homology rate is 0.38, scored 18.45, the threshold value is 13.30.

### EXAMPLE 3

### Cloning ATP-Dependent Helicase Protein 68 Gene by RT-PCR

The template was total RNA extracted from embryonic brain. The reverse transcription was carried out with oligo-dT primer to produce cDNA. After cDNAs were purified with Qiagen Kit, PCR was carried out with the following primers:

Primer1: 5′-GGGGGAAGTGGATTCTACGCTCCG-3′ (SEQ ID NO:3)

Primer2: 5′-GAAAAGAAAACACTGTCTTTTATT-3′ (SEQ ID NO:4)

Primer1 is the forward sequence started from position 1 bp of the 5′ end of SEQ ID NO: 1.

Primer2 is the reverse sequence of the 3′ end of SEQ ID NO: 1.

The amplification condition was a 50 μl reaction system containing 50 mmol/L KCl, 10 mmol/L Tris-Cl (pH 8.5), 1.5 mmol/L MgCl₂, 200 μmol/L dNTP, 10 pmol of each primer, 1U Taq DNA polymerase (Clontech). The reaction on a PE 9600 DNA amplifier (Perkin-Elmer) subjected to 94° C. 30 sec, 55° C. 30 sec, and 72° C. 2 min for 25 cycles. β-actin was used as a positive control, and a blank template as a negative control in RT-PCR. The amplified products were purified with QIAGEN kit, and linked with pCR vector (Invitrogen) using TA Cloning Kit. The DNA sequencing results show that the DNA sequence of PCR products was identical to nucleotides 1-2248 bp of SEQ ID NO: 1.

### EXAMPLE 4

### Northern Blotting of Expression of ATP-Dependent Helicase Protein 68 Gene

The total RNA was extracted by one-step method (Anal. Biochem 1987, 162, 156-159) with guanidinium isocyanate-phenol-chloroform. That is, homogenize the tissue using 4M guanidinium isocyanate-25 mM sodium citrate and 0.2M sodium acetate (PH4.0), add 1 volume phenol and ⅕ volume chloroform-isoamyl alcohol (49:1), centrifuge after mixing. The water phase was removed, 0.8 volume isopropyl alcohol was added, then the mixture was centrifuged. The RNA precipitation pellet was washed using 70% ethanol, then dried, then dissolved in water. 20 μg RNA was electrophoresed on 1.2% agarose gel containing 20 mM 3-(N-morpholino) propane sulfonic acid (pH 7.0)-5 mM sodium acetate-1 mM EDTA-2.2M formaldehyde; then transferred to a nitrocellulose filter. α-³²P-labelled DNA probe with α-³²P dATP was prepared by random priming method. The DNA probe having the coding sequence (176 bp-2035 bp) of ATP-dependent helicase protein 68 was amplified by PCR and is indicated in FIG. 1. The nitrocellulose filter with the transferred RNA was hybridized with the α-³²P-labeled DNA probe (2×10⁶ cpm/ml) overnight at 42° C. in a buffer containing 50% formamide-25 mM (KH₂PO₄) (pH 7.4) 5×SSC−5× Denhardt's solution and 200 μg/ml salmine DNA. The filter was then washed in the 1×SSC−0.1% SDS, at 55° C., for 30 min, and then analyzed and quantitatively determined using Phosphor Imager.

### EXAMPLE 5

### In vitro Expression, Isolation and Purification of Recombinant ATP-Dependent Helicase Protein 68

A pair of primers for specific amplification was designed based on SEQ ID NO: 1 and the encoding region in FIG. 1. The sequences are as follows:

Primer3: 5′-CCCCATATGATGTTTGTTCCAAGATCTCTAAAA-3′ (Seq ID No:5)

Primer4: 5′-CCCGAATTCTCATTTCTGAGAATTACTTTTATC-3′ (Seq ID No:6)

These two primers contain a NdeI and EcoRI cleavage site on the 5′ end respectively. After the sites are the coding sequences of the 5′ and 3′ ends of the desired gene. NdeI and EcoRI cleavage sites correspond to the selective cleavage sites on the expression vector pET-28 (+) (Novagen, Cat. No. 69865.3). PCR amplification was performed with the plasmid pBS-0577G09 containing the full-length target gene as a template. The PCR reaction was subject to a 50 μl system containing 10 pg pBS-0577G09 plasmid, 10 pmol of Primer-3 and 10 pmol of Primer-4, and 1 μl of Advantage polymerase Mix (Clontech). The parameters of PCR were 94° C. 20 sec, 60° C. 30 sec, and 68° C. 2 min for 25 cycles. After digesting the amplification products and the plasmid pET-28(+) by NdeI and EcoRI respectively, the large fragments were recovered and ligated with T4 ligase. The ligated product was transformed into E. coli DH5α with the calcium chloride method. After culturing overnight on a LB plate containing a final concentration of 30 μg/ml kanamycin, positive clones were selected using colony PCR and then sequenced. The positive clone (pET0577G09) with the correct sequence was selected and the recombinant plasmid was transformed into E. coli BL21(DE3)plySs (Novagen) using the calcium chloride method. In a LB liquid medium containing a final concentration of 30 μg/ml of kanamycin, the host bacteria BL21(pET-0577G09) were cultured at 37° C. to the exponential growth phase, then IPTG was added to the final concentration of 1 mmol/L, the cells were cultured for another 5 hours, and then centrifuged to harvest the bacteria. Afterwards the bacteria were sonicated, the supernatant was collected by centrifugation. Then the purified desired protein—ATP-dependent helicase protein 68-was obtained by a His.Bind Quick Cartridge (Novagen) affinity column binding 6His-Tag. SDS-PAGE showed a single band at 68 kDa (FIG. 2). The band was transferred onto a PVDF membrane and the N terminal amino acid was sequenced by Edams Hydrolysis, which showed that the first 15 amino acids on N-terminus were identical to those in SEQ ID NO: 2.

### EXAMPLE 6

### Prodcution of Antibody Against ATP-Dependent Helicase Protein 68

The following specific ATP-dependent helicase protein 68 polypeptide was synthesized by a polypeptide synthesizer (PE-ABI): NH2-Met-Phe-Val-Pro-Arg-Ser-Leu-Lys-Ile-Lys-Arg-Asn-AlaAsn-Asp-COOH (SEQ ID NO:7). The polypeptide was conjugated with hemocyanin and bovine serum-albumin (BSA) respectively to form two composites (See Avrameas et al., Immunochemistry, 1969, 6:43). 4 mg of hemocyanin-polypeptide composite was used to immunize rabbits together with Freund's complete adjuvant. The rabbits were re-immunized with the hemocyanin-polypeptide composite and Freund's incomplete adjuvent 15 days later. The titer of antibody in the rabbit sera was determined with a titration plate coated with 15 μg/ml BSA-polypeptide composite by ELISA. The total IgG was isolated from the sera of an antibody positive rabbit with Protein A-Sepharose. The polypeptide was bound to Sepharose 4B column activated by cyanogen bromide and the antibodies against the polypeptide were isolated from the total IgG by affinity chromatography. The immunoprecipitation analysis demonstrated that the purified antibodies specifically bind to ATP-dependent helicase protein 68.

### EXAMPLE 7

### Application of the Polynucleotide Fragments of Said Invention as Hybrid Probes

Selection of suitable oligonucleotides from the polynucleotide of said invention as hybrid probes can be versatilly applied. The said probe could be used to determine the existence of polynucleotide of said invention or its homologous polynucleotide sequences by hybridization with genome, or cDNA library of normal or clinical tissues from varied sources. The said probes could be further used to determine whether polynucleotide of said invention or its homologous polynucleotide sequences are abnormally expressed in cells from normal or clinical tissues.

Object of the following example is to select suitable oligonucletide fragments from the said invented polynuleotide SEQ ID NO:1 as hybrid probes to apply in membrane hybridization to determine whether there are polynucleotide of said invention or its homologous polynucleotide sequences in examined tissues. Membrane hybridization methods include dot hybridization, Southern blot, Northern blot, and replica hybridization. All these methods follow nearly the same steps after the polynucleotide samples are immobilized on membranes. These same steps are: membranes with samples immobilized on are prehybridized in hybrid buffer not containing probes to block nonspecific binding sites of the samples on membranes. Then prehybrid buffer is replaced by hybrid buffer containing labeled probes and continue incubation at the appropriate temperature so probes hybridize with the target nucleotides. Free probes are washed off by a series of washing steps after the hybrid step. A high-stringency washing condition (relatively low salt concentration and high temperature) is applied in the said example to reduce the hybridization background and remain highly specific signal. Two types of probes are selected for the said example: the first type probes are oligonucleotides identical or annealed to the said invented polynucleotide SEQ ID NO:1; the second type probes are oligonucleotides partially identical or partially annealed to the said invented polynucleotide SEQ ID NO:1. Dot blot method is applied in the said example for immobilization of the samples on membrane. The strongest specific signal produced by hybridization between first type probes and samples is remained after relatively strict membrane washing steps.

### Selection of Probes

The principles below should be followed and some things should be taken into consideration for selection of oligonucleotide fragments from the said invented polynucleotide SEQ ID NO:1 as hybrid probes:

1. the optimal length of probes should be between eighteen and fifty nucleotides.

2. GC amount should be between 30% and 70%, since nonspecific hybridization increases when GC amount is more than 70%.

3. there should be no complementary regions within the probes themselves.

4. probes meeting to the requirements above could be initially selected for further computer-aided sequence analysis, which includes homology comparison between said initial selected probes and its sourced sequence region (SEQ ID NO: 1), other known genomic sequences and their complements. Generally, said initial selected probes should not be used when they share fifteen identical continuous base pairs, or 85% homology with non-target region.

5. whether said initial selected probes should be chose for final application relies on further experimental confirmation.

The following two probes could be selected and synthesized after the analysis above:

Probe one belongs to the first type probes, which is completely identical or annealed to the gene fragments of SEQ ID NO: 1(41 Nt):

5′-TGTTTGTTCCAAGATCTCTAAAAATCAAGAGGAATGCTAAT-3′ (SEQ ID NO: 8)

Probe two belongs to the second type probes which is a replaced or mutant sequence of the gene fragments of SEQ ID NO: 1, or of its complementary fragments (41 Nt):

5′-TGTTTGTTCCAAGATCTCTAGAAATCAAGAGGAATGCTAAT-3′ (SEQ ID NO: 9),

Any other frequently used reagents unlisted but involved in the following concrete experimental steps and their preparation methods can be found in the reference: DNA PROBES G. H. Keller; M. M. Manak; Stockton Press, 1989 (USA) or a more commonly used molecular cloning experimental handbook Molecular Cloning (J. Sambrook et al. Acadimic press, 1998, 2nd edition).

### Sample Preparation

1, Extract DNA From Fresh or Frozen Tissues

Steps: 1) move the fresh or newly thawy tissue (source tissue of said invented polyucleotide) onto a ice-incubated dish containing phosphate-buffered saline (PBS). Cut the tissue into small pieces with scissors or a operating knife. Tissue should be remained damp through the operation. 2) mince the tissue by centrifugation at 1,000 g for 10 minutes. 3) resuspend the pellet (about 10 ml/g) with cold homogenating buffer (0.25 mol/l saccharose; 25 mmol/l Tris-HCl, pH 7.5; 25 mmol/L NaCl; 25 mmol/L MgCl₂) 4) homogenate tissue suspension at 4° C. full speed with an electronic homogenizer until it's completely smashed. 5) centrifuge at 1,000 g for 10 minutes. 6) resuspend the cell pellet (1-5 ml per 0.1 g initial tissue sample), and centrifuge at 1,000 g for 10 minutes. 7) resuspend the pellet with lysis buffer (1 ml per 0.1 g initial tissue sample), and continue to use the phenol extraction method.

2, Phenol Extraction of DNA

steps: 1) wash cells with 1-10 ml cold PBS buffer and centrifuge at 1000 g for 10 minutes. 2) resuspend the precipitated cells with at least 100 μl cold cell lysis buffer (1×10⁸ cells/ml). 3) add SDS to a final concentration of 1%. Addition of SDS into the cell precipitation before cell resuspension will cause the formation of large balls by cells which is difficult to be smashed and total production will be reduced. This phenomenon is especially severe when extracting more than 10⁷ cells. 4) add proteinase k till the final concentration reaches 200 μg/ml. 5) incubate at 50° C. for an hour or shake gently overnight at 37° C. 6) add an equal volume of phenol:chloroform:isoamyl alcohol (25:24:1) to the DNA solution to be purified in a microcentrifuge tube, and centrifuge for 10 minutes. If the two phases are not clearly separated, the solution should be recentrifuged. 7) remove the water phase to a new tube. 8) add an equal volume of chloroform:isoamyl alcohol (24:1) and centrifuge for 10 minutes. 9) remove the water phase containing DNA to a new tube and then purify DNA by ethanol precipitation.

3, DNA Purification by Ethanol Precipitation

steps: 1) add 1/10 vol of 2 mol/L sodium acetate and 2 vol of cold 100% ethanol into the DNA solution, mix and place at −20° C. for an hour or overnight. 2) centrifuge for 10 minutes. 3) Carefully suck out or pour out the ethanol. 4) add 500 μl of cold 70% ethanol to wash the pellet and centrifuge for 5 minutes. 5) carefully suck out or pour out the ethanol, add 500 μl of cold ethanol to wash the pellet and centrifuge for 5 minutes. 6) carefully suck out or pour out the ethanol and invert the tube on bibulous paper to remove remnant ethanol. Air dry for 10-15 minutes to evaporate ethanol on pellet surface. But notice not to dry the pellet completely since completely dry pellet is difficult to be dissolved again. 7) resuspend the DNA pellet with a small volume of TE or water. Spin at low speed or blow with a drip tube, and add TE gradually and mix until DNA is completely dissolved. Nearly add 1 μl TE every 1−5×10⁶ cells.

The following 8-13 steps are applied only when contamination must be removed, otherwise go to step 14 directly. 8) add Rnase A into DNA solution to a final concentration of 100 μg/ml and incubate at 37° C. for 30 minutes. 9) add SDS and protease K to the final concentration of 0.5% and 100 μg/ml individually, and incubate at 37° C. for 30 minutes. 10) add an equal volume of phenol:chloroform:isoamyl alcohol (25:24:1), and centrifuge for 10 minutes. 11) carefully remove out the water phase and extract it with an equal volume of chloroform:isoamyl alcohol (24:1) and centrifuge for 10 minutes. 12) carefully remove out the water phase, and add 1/10 vol of 2 mol/L sodium acetate and 2.5 vol of cold ethanol, then mix and place at −20° C. for an hour. 13) wash the pellet with 70% ethanol and 100% ethanol, air dry and resuspend DNA as same as the steps 3-6. 14) determine the purity and production of DNA by A₂₆₀ and A₂₈₀ assay. 15) separate DNA sample into several portions and store at −20° C.

### Preparation of Sample Membrane

1) Take 4×2 pieces of nitrocellulose membrane (NC membrane) of desired size, and lightly mark out the sample dot sites and sample number with a pencil. Every probe needs two pieces of NC membrane, so then membranes could be washed under high stringency condition and stringency condition individually in the following experimental steps.

2) Cuck 15 μl of samples and control individually, dot them on the membrane, and dry at room tempreture.

3) Place the membranes on filter paper soaked in 0.1 mol/L NaOH, 1.5 mol/L NaCl, leave for 5 minutes (twice), and allow to dry. Transfer the membranes on filter paper soaked in 0.5 mol/L Tris-HCl (pH 7.0) 3 mol/L NaCl, leave for 5 minutes (twice), and allow to dry.

4) Place the membranes between clean filter paper, packet with aluminum foil, and vacuum dry at 60-80° C. for 2 hours.

### Labeling of Probes

1) Add 3 μl probe (0.10D/10 μl), 2 μl kinase buffer, 8-10 uCi γ-³²P-dATP+2U Kinase, and add water to the final volume of 20 μl.

2) Incubate at 37° C. for 2 hours.

3) Add ⅕ vol bromophenol blue indicator (BPB).

4) Load that sample on Sephadex G-50 column.

5) Collect the first peak before the elution of ³²P-Probe (monitor the eluting process by Monitor).

6) Five drops each tube and collect for 10-15 tubes.

7) Measure the isotope amount with liquid scintillator.

8) Merged collection of the first peak is the prepared ³²P-Probe (the second peak is free γ-³²P-dATP)

### Prehybridization

Place the sample membranes in a plastic bag, add 3-10 mg prehybrid buffer (10× Denhardt's; 6×SSC, 0.1 mg/ml CT DNA (calf thymus gland DNA)), seal the bag, and shake on a 68° C. water bath for two hours hybridization.

Cut off a corner of the plastic bag, add in prepared probes, seal the bag, and shake on a 42° C. water bath overnight.

### Membrane Washing

Membrane washing applying a high-stringency condition:

1) Take out the hybridized sample membranes.

2) Wash the membranes with 2×SSC, 0.1% SDS at 40° C. for 15 minutes (twice).

3). Wash the membranes with 0.1×SSC, 0.1% SDS at 40° C. for 15 minutes (twice).

4) Wash the membranes with 0.1×SSC, 0.1% SDS at 55° C. for 30 minutes (twice), and dry at room temperature.

Membrane washing applying a low-stringency condition:

1) Take out the hybridized sample membranes.

2) Wash the membranes with 2×SSC, 0.1% SDS at 37° C. for 15 minutes (twice).

3) Wash the membranes with 0.1×SSC, 0.1% SDS at 37° C. for 15 minutes (twice).

4) Wash the membranes with 0.1×SSC, 0.1% SDS at 40° C. for 15 minutes (twice), and dry at room temperature.

X ray autoradiography:

X ray autoradiograph at −70° C. (autoradiograph time varies according to radioactivity of the hybrid spots).

### Experimental Results

In hybridization experiments carried out under low-stringency membrane washing condition, the radioactivity of all the above four probes hybrid spots shows no obvious difference; while in hybridization experiments carried out under high-stringency membrane washing condition, radioactivity of the hybrid spot by probe one is obviously stronger than the other three's. So probe one could be applied in qualitative and quantitive analysis of the existence and differential expression of said invented polynucleotide in different tissues.

### EXAMPLE 8

### DNA Microarray

DNA-chip or DNA Microarray technology is now studied and developed in many laboratories and large pharmaceutical companies. The technology uses a lot of target genes that are arrayed on a glass or silicon slide with high density, then uses fluorescence detection and software to compare and analyze the data. So it can analyze a large-scale of biology information quickly and effectively. The polynucleotide provided in this invention may be used to find new gene function as target DNA by DNA-chip technology, screen tissue-specific gene especially tumor related gene, disease (hereditary diseases etc.) diagnosis. The methods have been explained in many documents (see, DeRisi, J. L., Lyer, V. & Brown, P.O. (1997) Science 278, 680-686; Helle, R. A., Schema, M., Chai, A., Shalom, D., (1997) PNAS 94:2150-2155.)

(I) DNA Fixation

4000 polyneucleotide sequences of different full-length cDNAs were taken as target DNA, including the polyneucleotide of this invention. cDNAs were amplified by PCR (as discussed in example 3 of the present invention) and purified, then adjusted to about 500 ng/μl. PCR products were printed on glass slide using Cartesian 7500 Robotics (Cartesian, USA), the gap is 280 μm.

Printed arrays were hydrated, dried, UV cross-linked, rinsed and dried to fix the DNA on the glass slide. The details of the method have been reported in many documents. The step after fixation in this example is:

1. incubate for 4 hrs in a humid chamber;

2. wash 1 min in 0.2% SDS;

3. wash 2×1 min in ddH₂O;

4. block for 5 min with NₐBH₄;

5. incubate in water at 95° C. for 2 mins;

6. wash 1 min in 0.2% SDS;

7. wash twice with ddH₂O;

8. air dry and store in the dark at 25° C.

(II) Probe Labeling

Total mRNA was extracted from normal liver and liver cancer by one step method, then purified using Oligotex mRNA Midi Kit (Qiagen). Following the reverse transcriptase step, mRNA from normal liver was labeled with Cy3dUTP (5-Aminopropargyl-2′-deoxyuridine 5′-triphate coupled to Cy3 fluorescent dye.purchased from Amersham Phamacia Biotech co.) and mRNA from liver cancer was labeled with Cy5dUTP (5-Amino-propargyl-2′-deoxyuridine 5′-triphate coupled to Cy5 fluorescent dye.purchased from Amersham Phamacia Biotech Co.). Then labeled probes were purified. (See Schena, M., Shalon, D., Heller, R. (1996) Proc. Natl. Acad. Sci. USA. Vol.93: 10614-10619; Schena, M., Shalon, Dari., Davis, R. W. (1995) Science. 270.(20):467-480.)

(III) Hybridization

Labeled probes from each tissue mixed with DNA-chip in UniHyb™ Hybridization Solution (TeleChem) for 16 hr. After washed with washing buffer (1×SSC, 0.2%SDS) at room temperature, Arrays were scaned using ScanArray 3000 (General Scanning Co., USA). The images were analyzed with Imagene (Biodiscovery Co., USA). The ratios of Cy3 to Cy5 were obtained. If the ratio less than 0.5 or larger than 2, we can conclude the gene was expressed differently in two tissues.

The results showed that Cy3 signal=6341.5 (average of four experiments), Cy5 signal=8087.95 (average of four experiments), Cy3/Cy5=0.7841. So there is no obvious differential expression in the two tissues of the polynucleotide provided in this invention.

1. An isolated polypeptide -ATP-DEPENDENT HELICASE PROTEIN 68 comprising a polypeptide having the amino acid sequence of SEQ ID NO: 2, its active fragments, analogues and derivatives.

2. The polypeptide of claim 1 wherein amino acid sequences of said polypeptide, its analogues or derivatives have at least 95% identity with the amino acid sequence of SEQ ID NO: 2.

3. The polypeptide of claim 2 wherein said polypeptide is a polypeptide comprising the amino acid sequence of SEQ ID NO: 2.

4. An isolated polynucleotide consisting one of:
(a) the polynucleotide encoding a polypeptide having an amino acid sequence of SEQ ID NO: 2 or its fragment, analogue, derivative;
(b) the polynucleotide complementary to polynucleotide (a); or
(c) a polynucleotide that shares at least 70% homology to the polynucleotide (a) or (b).

5. The polynucleotide of claim 4 comprising a polynucleotide encoding an amino acid sequence of SEQ ID NO: 2.

6. The polynucleotide of claim 4 wherein the sequence of said polynucleotide comprises position 176-2035 of SEQ ID NO: 1 or position 1-2248 of SEQ ID NO: 1.

7. A recombinant vector containing an exogenous polynucleotide which is constructed with the polynucleotide of any of claims 4-6 and plasmid, virus, or expression vector.

8. A genetically engineered host cell containing an exogenous polynucleotide which is selected from:
(a) the host cell transformed or transfected by the recombinant vector of claim 7; or
(b) the host cell transformed or transfected by the polynucleotide of any of claims 4-6.

9. A method for producing a polypeptide having the activity of ATP-dependent helicase protein 68, which comprises the steps of:
(a) culturing the engineered host cell of claim 8 under the conditions suitable for expression of ATP-dependent helicase protein 68;
(b) isolating the polypeptides having the activity of ATP-dependent helicase protein 68 protein from the culture.

10. An antibody specifically which binds bound specifically with ATP-dependent helicase protein 68.

11. A compound simulating or regulating the activity or expression of the polypeptide which is the compound simulating, improving, antagonizing, or inhibiting the activity of ATP-dependent helicase protein 68.

12. The compound of claim 11 which is an antisense sequence of the polynucleotide sequence of SEQ ID NO: 1 or its fragment.

13. The use of the compound of claim 11 for regulating the activity of ATP-dependent helicase protein 68 in vivo or in vitro.

14. A method for detecting a disease related to the polypeptide of any of claims 1-3 or susceptibility thereof which comprises detecting the amount of expression of said polypeptide, or detecting the activity of said polypeptide, or detecting the nucleotide variant of the polynucleotide causing said abnormal expression or activity.

15. The use of the polypeptide of any of claims 1-3 for screening the mimetics, agonists, antagonists or inhibitors of ATP-dependent helicase protein 68; or for the identification of peptide spectrum.

16. The use of the nucleic acid molecule of any of claims 4-6 wherein it is used as primer in the nucleic acid amplification, or as probe in the hybridization reaction, or is used for manufacture of gene chip or microarray.

17. The use of the polypeptide, polynucleotide or compound of any of claims 1-6 and 11 wherein a safe and effective amount of said polypeptide, polynucleotide or its mimetics, agonists, antagonists or inhibitors are mixed with the pharmaceutically acceptable carrier to form the pharmaceutical composition for the diagnosis or treatment of diseases associated with the abnormality of ATP-dependent helicase protein 68.

18. The use of the polypeptide, polynucleotide or compound of any of claims 1-6 and 11 wherein said polypeptide, polynucleotide or compound are used for the manufacture of medicine for the treatment of malignant tumors, haemal disease, HIV infection and immune system diseases and various inflammation.
